(19) United States
(12) Patent Application Publication
(10) Pub. No.: US 2026/0033892 A1
(43) Pub. Date: Feb. 5, 2026

(54) BALLOON-BASED DEVICE FOR CREATING INTERATRIAL SHUNT

(71) Applicant: MEDTRONIC, INC., Minneapolis, MN (US)

(72) Inventors: Nicolas Coulombe, Anjou (CA); Kevin R. Seifert, Forest Lake, MN (US); Neal C. Duong, Ramsey, MN (US)

(21) Appl. No.: 19/150,032

(22) PCT Filed: Jan. 17, 2024
(86) PCT No.: PCT/IB2024/050458

§ 371 Date: Jul. 22, 2025

Related U.S. Application Data
(60) Provisional application No. 63/481,134, filed on Jan. 23, 2023.

(51) Int. Cl. A61B 18/14; A61B 18/00; A61B 18/02

(52) U.S. Cl. CPC A61B 18/1492; A61B 18/02; A61B 18/1487; A61B 2018/00023; A61B 2018/00077; A61B 2018/0022; A61B 2018/00351; A61B 2018/00577; A61B 2018/00601; A61B 2018/00625; A61B 2018/00678; A61B 2018/00714; A61B 2018/0212

(57) ABSTRACT

A medical device for creating an interatrial shunt between atria of a patient's heart is disclosed. The device includes an elongated support member, an inflatable balloon, and a plurality of electrical conductors. The inflatable balloon is configured to fit inside a lumen of the elongated support member when deflated. and can be advanced out of the distal portion of the elongated support member. The electrical conductors extend longitudinally along the balloon for delivering electrical energy to the atrial septum of the patient. The electrical conductors further are radially spaced apart from one another to form a multicuspid opening in the atrial septum.

This application claims the benefit of U.S. Provisional Patent Application Ser. No. 63/481,134, filed Jan. 23, 2023, the entire content of which is incorporated herein by reference.

### TECHNICAL FIELD

This disclosure generally relates to medical devices and, more particularly, to medical devices and associated techniques for forming shunts.

### BACKGROUND

Heart failure is a common syndrome in which a patient's heart output is insufficient to meet the body's needs. When a patient suffers from some forms of heart failure, the pressure in the left atrium may be higher than desired. To relieve the pressure in the left atrium, surgeons may use an implanted device such as a stent to create a shunt between the left and right atria. This interatrial shunting procedure can decompress the left atrium by creating a blood flow pathway between the right atrium and left atrium. Recent effort has been directed to procedures that create an interatrial shunt without the need for an implant. These implant-less procedures eliminate the risk of implant failure and provide for continued access to the left atrium for future procedures. In some such implant-less procedures, a puncture is created in the interatrial septum and the tissue surrounding the puncture is ablated with thermal energy, such as cryogenic energy to prevent tissue regrowth and to maintain the shunt.

### SUMMARY

The present disclosure describes systems, devices, and techniques for creating a fluid pathway, or shunt, between the left atrium and right atrium of a heart of a patient. The shunt can be used, for example, to treat patients having heart failure and/or pulmonary edema. While typical shunting procedures may result in tissue overgrowth, thus reducing the effectiveness of the shunt, in examples described herein, tissue of the interatrial septum may be ablated, reducing risk of such overgrowth. According to the present disclosure, a medical system includes a cutting tool configured to puncture through a target treatment site and cut septal wall tissue to form a multi-cuspid valvular shunt in the septal wall, and an ablation device configured to ablate septal wall tissue of at least a portion of the multi-cuspid valvular shunt to make the multi-cuspid valvular shunt biostable, e.g., to inhibit overgrowth, scarring, and/or attachment of the cut portions of the septal wall tissue to reduce and/or prevent the multi-cuspid valvular shunt from closing. A multi-cuspid valvular shunt may be configured to open to relieve left-ventricular pressure, but at other times remain closed for improved cardiac functioning relative to conventional interatrial shunts.

While an interatrial shunting procedure has become more common, it remains a difficult and costly procedure that limited patients can access. In some aspects, the present disclosure provides a precise surgical tool and procedure for making a biostable, multi-cuspid valvular shunt to, for example, treat heart failure, making the procedure available to more patients and offering those patients a better outcome.

In some examples, a medical device includes an elongated support member, an inflatable balloon at a distal portion of the elongated support member, and a plurality of electrical conductors extending longitudinally along the balloon. The plurality of conductors are configured to deliver electrical energy to an atrial septum of a patient, the electrical conductors being radially spaced apart from one another to form a multicuspid opening in the atrial septum by delivery of the electrical energy.

In some examples, a method includes guiding a surgical tool to a heart of a patient, inflating at least one balloon at a distal end of the surgical tool, energizing a plurality of electrical conductors that extend longitudinally along the balloon, the electrical conductors being radially spaced apart from one another on a surface of the at least one balloon, and forming a multicuspid opening in an atrial septum of the patient by delivering electrical energy to the atrial septum via the electrical conductors.

This summary is intended to provide an overview of the subject matter described in this disclosure. It is not intended to provide an exclusive or exhaustive explanation of the apparatus and methods described in detail within the accompanying drawings and description below. The details of one or more aspects of the disclosure are set forth in the accompanying drawings and the description below.

### BRIEF DESCRIPTION OF DRAWINGS

FIG. 1 is an illustration of an example of a portion of a surgical tool for creating an interatrial shunt according to some aspects of the disclosure.

FIGS. 2A-2B are a distal end-view illustrations of examples of the surgical tool of FIG. 1.

FIG. 3 is an illustration of an example of a portion of another surgical tool for creating an interatrial shunt according to some aspects of this disclosure.

FIG. 4 is an illustration of an example of a portion of another surgical tool for creating an interatrial shunt according to some aspects of this disclosure.

FIGS. 5A-5B are illustrations of an example of a portion of another surgical tool for creating an interatrial shunt according to some aspects of this disclosure.

FIG. 6 is an illustration of an example of a portion of another surgical tool for creating an interatrial shunt according to some aspects of this disclosure.

FIG. 7 is a flow chart illustrating an example of a process for creating an interatrial shunt according to some aspects of this disclosure.

FIG. 8 is a flow chart illustrating another example of a process for creating an interatrial shunt according to some aspects of this disclosure.

FIG. 9 is a schematic illustration of a surgical system for creating an interatrial shunt according to some aspects of this disclosure.

FIG. 10A is a flow chart illustrating an example of a process for ablating cardiac tissue according to some aspects of this disclosure.

FIG. 10B is a flow chart illustrating an example of a process for cooling electrical conductors according to some aspects of this disclosure.

### DETAILED DESCRIPTION

The present disclosure describes examples of medical systems, devices, and techniques for creating a fluid pathway, or shunt, between the left atrium and right atrium of a heart of a patient without the use of an implant such as a stent, to maintain the shunt. The shunt may be formed as a valve, e.g., a multi-cuspid valve, in a septal wall between the left and right atrium, and at least a portion of the valve may be ablated. A surgical tool includes RF ablation electrodes that facilitate cutting the atrial septum in a particular pattern that results in a multi-cuspid valve-like opening in the atrial septum. The electrodes are coupled with a balloon structure that facilitates electrode deployment in a patient's heart. In some examples, the balloon is a cryoballoon that enables cryoablation of the tissue in the atrial septum to stabilize the shunt.

In accordance with example medical systems, devices, and techniques described herein, septal wall tissue may be ablated via delivering energy (e.g., radiofrequency (RF) energy, plasma energy, or the like), and/or via cryoablation (e.g., via a cryogenic device such as a cryogenic balloon) to ablate a portion of the valve, e.g., septal wall tissue forming the valve. Due to the nature of the ablation, the tissue adjacent to the ablation will fibrose/endothelialize and define an opening (e.g., a shunt) which may be formed as a multi-cuspid valve between the left atrium and the right atrium, enabling pressure from the left atrium to decompress into the right atrium. This may help treat heart failure and/or pulmonary edema, such as by mitigating a mechanism of heart failure and/or pulmonary edema. In other examples, the systems, devices, and techniques described herein can be used to create a shunt between two other hollow anatomical structures of a patient and to treat other patient conditions. Thus, while a shunt between a left atrium of a heart of a patient and a right atrium of the patient is primarily referred to herein, the systems, devices, and techniques can be used to form shunts in other locations of the heart, other locations of the body of patients, or for other medical procedures in other examples.

Some example medical systems, devices, and techniques include ablating at least a portion of the valve to stabilize and/or make the valve biostable, e.g., to prevent tissue overgrowth of the septal wall in the process of wound healing that leads to fusing together and closing of the valve. For example, medical systems, devices, and techniques include ablating to cause scarring, lesions, or the like, that prevent the tissue from fusing together and closing the valve. In some examples, medical systems, devices, and techniques include ablating at least a portion of the valve to stabilize and/or make the valve biostable for a period of time, e.g., after underlying causes of a pressure differential between left and right atria are removed and/or eliminated and a shunt is no longer needed. For example, after a period of time, the pressure differential between the left and right atria may reduce, and the biostabilized leaflets, tissue flats, and/or cuspids may be in contact for longer periods of time, which may promote fusing. In other examples, after the period of time and reduction of the pressure differential, surgical and/or pharmaceutical treatments may be used to further promote fusing.

The techniques of this disclosure can be used to treat certain forms of heart failure, such as heart failure with preserved ejection fraction. For instance, forming a shunt between the left atrium and the right atrium with the systems and devices described herein can enable the relief of pressure in the left atrium of a patient without requiring the permanent implantation of a foreign object (e.g., a stent or the like), leading to better patient outcomes. In addition, the systems and devices described herein are highly user-friendly, e.g., do not require extensive training for the clinician.

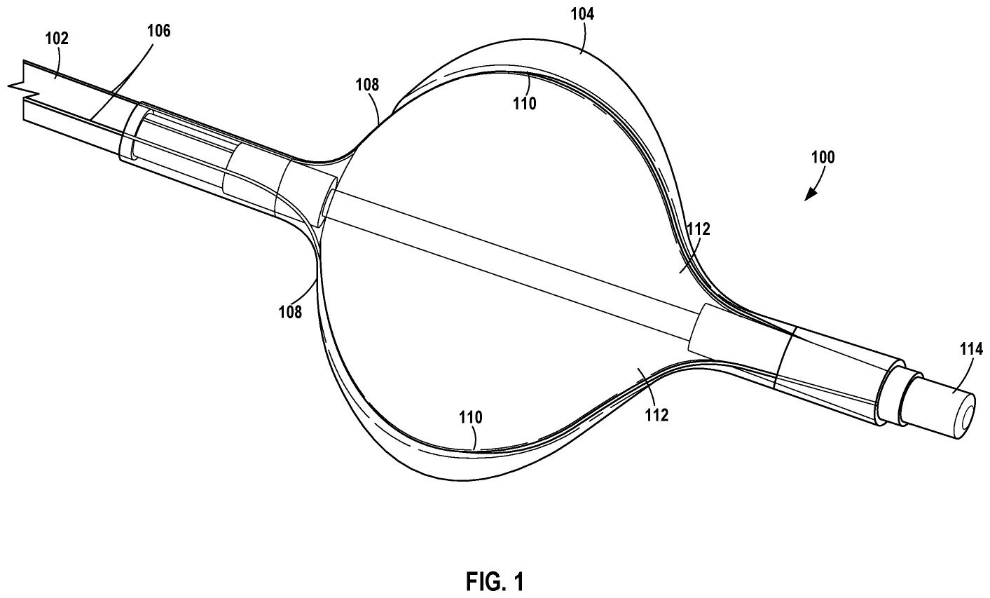

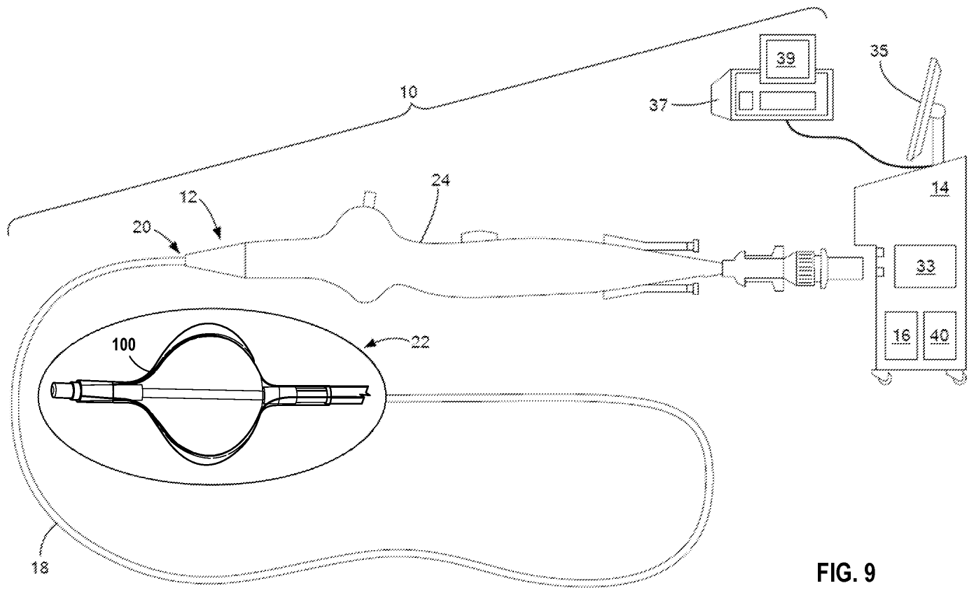

FIG. 9 is a schematic illustration of a surgical system 10 for creating an interatrial shunt according to some aspects of this disclosure. Surgical system 10 includes a device 12 configured to be coupled with a control unit 14. Device 12 includes an elongate body 18, e.g., including a catheter and/or guidewire that can be steerable within a patient's circulatory system to a patient's heart. Device 12 further includes a proximal end 20 where a handle 24 is located, and a distal end 22 including a surgical tool 100, described below and with one example illustrated in FIG. 1.

Handle 24 includes one or more interfaces with control unit 14, which is illustrated with an optional user interface 35. For example, handle 24 may be coupled with fluid supply reservoir 16 and a fluid recovery reservoir or scavenging system 40. Fluid supply reservoir 16 may include any suitable number of fluid supply reservoirs for any suitable fluids, including, for example, a cooling fluid such as liquid nitrous oxide, and a non-cryogenic fluid such as air. According to an aspect of the disclosure, each reservoir of the fluid supply reservoirs 16 may separately deliver a respective fluid via a lumen of the elongate body 18 to the surgical tool 100, as described further below. Control unit 14 further includes a processing circuit 33 for controlling electronic operations of the system 10, and an interface with a generator 37 for generating electrical energy for, e.g., pulsed field ablation (PFA) and/or radio frequency (RF) ablation via surgical tool 100. In the illustrated example, PFA/RF generator 37 includes an optional user interface 39.

FIG. 1 illustrates a surgical tool 100 according to some exemplary aspects of this disclosure. FIG. 1 is a close-up view of a distal end 22 of the surgical tool 100 of FIG. 9. A surgical tool 100 according to some examples includes an elongated support member 102, an inflatable balloon 104, and a plurality of electrical conductors or electrodes 106. The elongated support member 102 is configured for delivery to a patient's heart via a steerable catheter or by being coupled to a guidewire (not illustrated). That is, in some examples, the elongated support member 102 may be configured to fit inside a lumen of a steerable catheter. In other examples, the elongated support member 102 may include a guidewire lumen extending through its length, such that the elongated support member 102 may be guided to the heart via guidewire. The elongated support member 102 includes a lumen coupled to the balloon such that the balloon 104 can be inflated via one of the fluid supply reservoirs 16 coupled to the proximal end 20 of the device 12. The electrical conductors 106 may be configured for RF/PFA ablation of tissue with which they come into contact. Furthermore, the balloon 104 may be configured for cryoablation of tissue with which it comes into contact.

When the balloon 104 is deflated, the balloon 104 and a portion of the electrical conductors 106 may be configured to fit inside a lumen of the elongated support member 102. For example, the deflated balloon 104 may be stowed in a delivery catheter of elongated support member 102. In another example, the deflated balloon 104 may be stowed in an integrated sheath or recess (not illustrated) in elongated support member 102. In this way, the elongated support member 102 may be guided from a point of entry, such as a patient's femoral vein, through the patient's circulatory system to the patient's heart. In an aspect of this disclosure, the surgical tool may be guided into a right atrium of a patient's heart as part of an interatrial shunting procedure.

Electrical conductors 106 extend at least a portion of the length of the elongated support member 102, being coupled at a proximal end of the elongated support member 102 to a suitable control unit (see FIG. 9) for activating the electrical conductors 106. In some examples, the electrical conductors 106 may be slidably engaged in grooves or lumen in the elongated support member 102. That is, when the balloon 104 inflates, the portion of the electrical conductors 106 in contact with the surface of the balloon 104 may deflect radially outward, and the electrical conductors 106 may slide inside grooves or lumens in the elongated support member 102. In another example, the electrical conductors 106 may be fixedly attached to the elongated support member 102, and the opposing ends 108/112 of the balloon 104 may be configured to move closer together as the balloon 104 inflates.

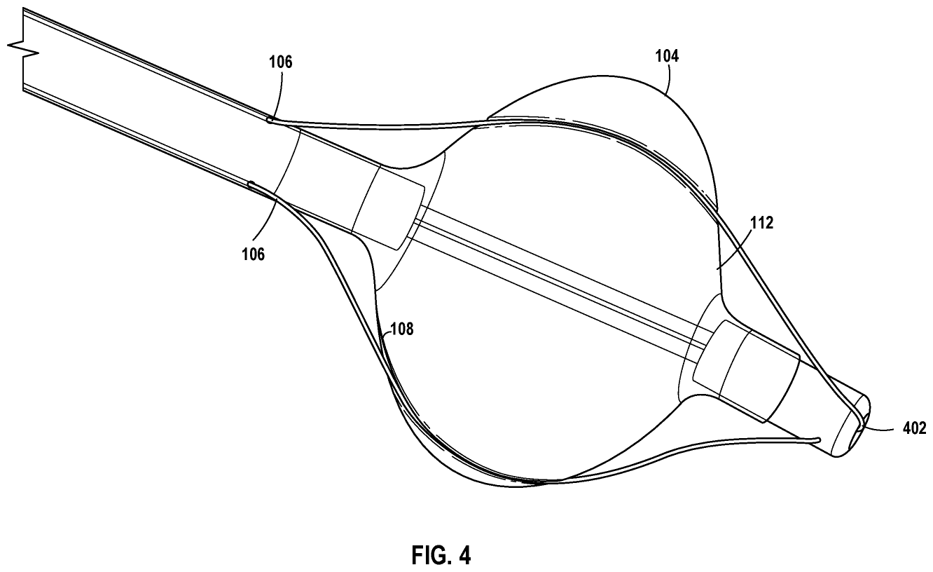

In the illustration of FIG. 1, the balloon 104 is inflated with a suitable fluid, such as but not limited to air, via fluid supply reservoir 16. Although other examples may differ (see, e.g., FIGS. 3 and 4 below), the electrical conductors 106 may extend longitudinally along the surface of the balloon 104, from its proximal end 108 to its distal end 112. The proximal portion of the electrical conductors 106, including the portion of the electrical conductors 106 at the elongated support member 102, as well as the portion on the proximal side of the balloon 104, may be insulated, or covered with a suitable electrical insulating material. For example, on the proximal side of the balloon 104 (i.e. the side of the proximal end 108 of the balloon 104), the electrical conductors 106 may be sandwiched between layers of a multi-layered balloon 104. In other examples, rather than being sandwiched between balloons, the electrical conductors 106 may be covered with a suitable insulating sheath at regions other than the cutting region.

As illustrated, the distal portion 112 of the electrical conductors 106 is a cutting region, which may be used for cutting. That is, the electrical conductors 106 emerge from their insulation at about a halfway point 110 across the surface of the balloon 104. Thus, at a distal end 112 of the balloon 104, the electrical conductors 106 may be exposed such that they may contact tissue.

In some examples described herein, an elongated support member 102 includes a puncture tool 114 configured to create an initial puncture through a septal wall between the left atrium and a right atrium. That is, after the elongated support member 102 is guided into the right atrium of the patient's heart, the elongated support member 102 may be advanced such that the puncturing tool 114 comes into contact with a target treatment site at the interatrial septum between the atria. In various examples, any suitable puncture tool 114 may be utilized, such as a tapered tip, a needle, an RF needle, a knife, etc. As one example, FIG. 4 illustrates an elongated support member wherein at least one of the electrical conductors 106 extends to a distal tip 402 of the surgical tool, showing that the electrical conductors 106 can, in some examples, perform as a puncture tool 114 in addition to a cutting tool. As another example, at least one of the electrical conductors 106 may transfer energy to a guidewire (not illustrated) within the catheter 102 to create the puncture.

Returning to FIG. 1, in the illustrated example, the distal end 112 of the balloon 104 is for cutting. That is, as the elongated support member 102 is advanced in the direction toward the target treatment site, after the puncture tool 114 has punctured the interatrial septum, the electrical conductors 106 may be energized. In this way, the electrical conductors 106 along the distal surface 112 of the balloon 104 may cut tissue via radio frequency ablation while the shape of the fully inflated balloon 104 expands the size of, or dilates the punctured opening.

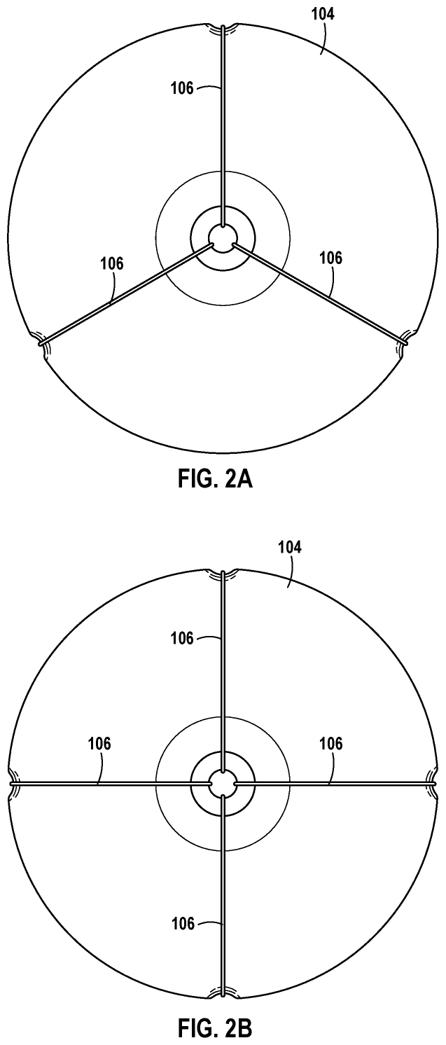

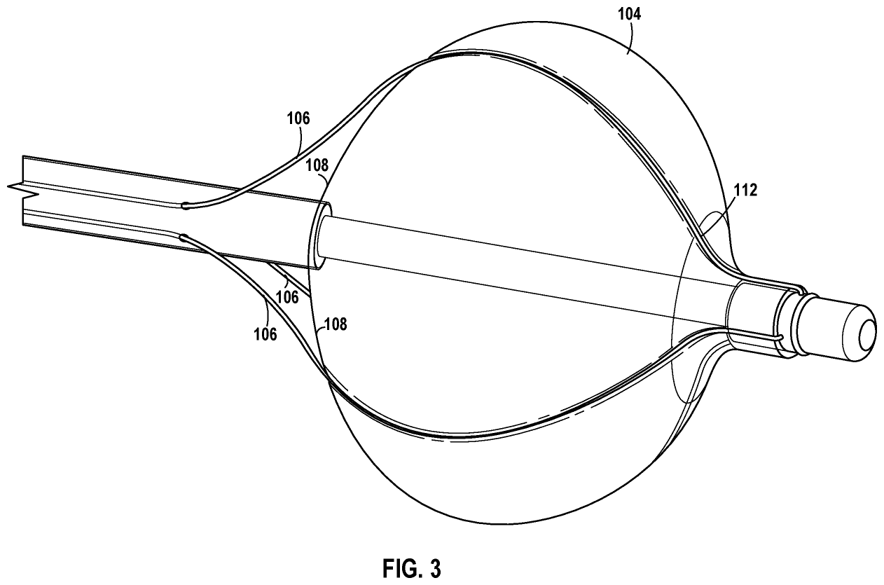

According to an aspect of this disclosure, the electrical conductors 106 may be radially spaced apart from one another to form a multicuspid opening in the atrial septum. FIGS. 2A and 2B illustrate two examples of surgical tools according to some aspects, where (FIG. 2A) three electrical conductors 106 are spaced at about 120 degrees apart, or where (FIG. 2B) four electrical conductors 106 are spaced at about 90 degrees apart. These figures illustrate examples of the surgical tool shown in FIG. 1, from a point of view directly from the distal tip. With these exemplary configurations, the electrical conductors 106 may cut the interatrial septum in a suitable shape to form a three-or four-leaf valve between the right and left atria. In various aspects, the present disclosure is not limited to the three or four conductors illustrated, and any suitable number of electrical conductors 106 for creating a multi-leaflet, multicuspid valvular opening may be used.

In various aspects, thermal cooling may be used for cryoablation and/or for cooling the electrical conductors 106. In some examples, the balloon 104 may be a cryoballoon. That is, the balloon 104 may be configured such that a suitable cooling fluid (e.g., a cryogenic fluid) may extract heat from cardiac tissue such as to cause ablation of the cardiac tissue. In some examples, the balloon 104 may be configured such that a suitable cooling fluid may extract heat from the electrical conductors 106. In these examples, the balloon need not necessarily be a cryoablation balloon, although the process can be followed similarly using a cryoablation balloon.

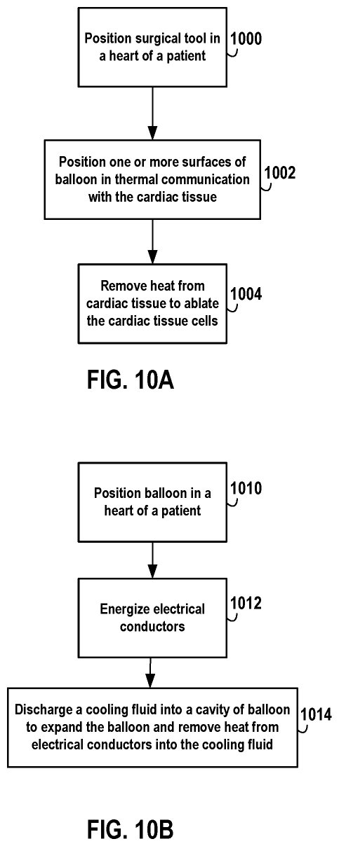

FIG. 10A is a flow chart of an example method for cryoablation in accordance with some aspects of the present disclosure. For instance, according to some aspects, after the electrical conductors 106 cut the multicuspid opening in the interatrial septum, a cryoablation procedure may be performed to ablate the tissue in the proximity to the newly formed opening. In this procedure, the balloon 104 may be at least partially filled with a suitable cryogenic fluid such that tissue in contact with the balloon 104 may be ablated. In this manner, the tissue forming the multicuspid opening between the right and left atria can be stabilized such that it is less likely to rapidly heal or overgrow new tissue.

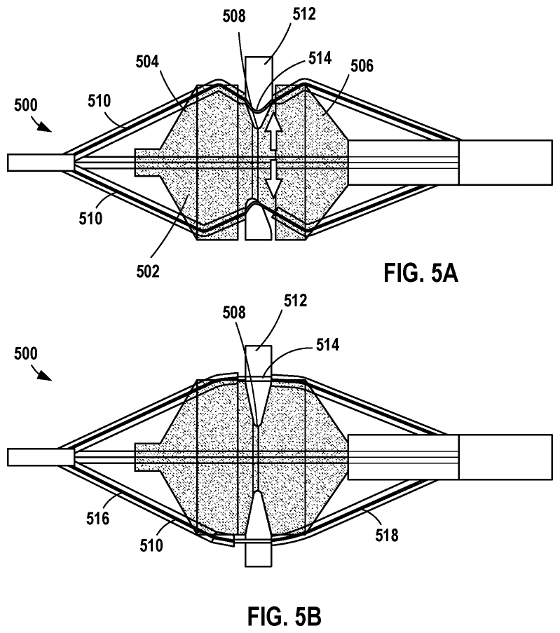

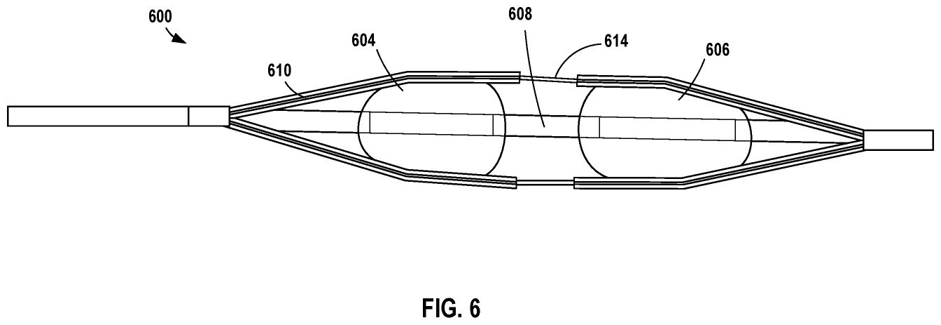

FIG. 10A will be described with respect to system 10 of FIG. 9, and may employ surgical tool 100 of any one or more of FIGS. 1-4, surgical tool 500 of FIG. 5, and/or surgical tool 600 of FIG. 6, but applies to other examples, systems, devices, and target hollow anatomical structures of a patient. The method of FIG. 10A includes positioning surgical tool 100 in a heart of a patient (1000). For example, a clinician may manipulate handle 24 and advance a distal end of surgical tool 100 to a target treatment site of an interatrial wall or septum of the patient, such that the balloon 104 is positioned proximate the target treatment site. With the aid of a medical imaging device, the clinician may confirm a position of surgical tool 100 in the heart.

The method of FIG. 10A includes positioning one or more surfaces of balloon 104 in thermal communication with the cardiac tissue (1002). As described in connection with FIGS. 1-4, surgical tool 100 may include a balloon 104 configured to expand such that one or more thermal surfaces of balloon 104 are in thermal communication with (e.g., contact or are proximate to) the cardiac tissue at the target treatment site. For example, the clinician may operate control unit 14 or fluid supply reservoir 16 to deliver a pressurized fluid to the balloon 104 to radially expand the balloon, operate an actuation mechanism to radially expand the balloon 104, or the like. In some examples, contacting the target treatment site with one or more surfaces of balloon 104 may include expanding the balloon 104 beyond an initial diameter of the elongate support member 102.

The method of FIG. 10A includes removing heat from the cardiac tissue (e.g., the atrial wall) to ablate the cardiac tissue cells (1004). To remove heat from the cardiac tissue, fluid supply reservoir 16 may be configured to control a cooling load delivered to surgical tool 100. For example, fluid supply reservoir 16 may receive various parameters, such as input by the clinician via secondary input 35, associated with an amount of cold therapy to be delivered to the target treatment site. To ablate the cardiac tissue, the heat is removed from the atrial wall to achieve and/or maintain a temperature of the cardiac tissue associated with ablation of the cardiac tissue. For example, the temperature of the cardiac tissue may be maintained within a target temperature range for a target therapy time range that corresponds to the cooling load. The target therapy time range can be, for example, stored in a memory of control unit 14 and/or generator 37 or another device. In some examples, the target tissue temperature range is defined by an upper temperature threshold value associated with death of the cardiac tissue. In some examples, the upper temperature threshold value is less than about 0° C.

In another example, according to some aspects, when the electrical conductors 106 are used to cut the patient's tissue, sufficient heat may be generated to damage the portion of the balloon 104 in contact with the electrical conductors 106. In other examples, radio frequency ablation cutting can vaporize the tissue and the surrounding blood, potentially creating bubbles in the patient's heart. Therefore, the cooling fluid may be applied to the balloon during the time when the electrical conductors 106 are cutting the tissue. In this way, the cooling fluid may cool the balloon 104 such that the electrical conductors 106 do not damage, melt, or burn the balloon 104. Further, the cooling fluid may cool the surrounding tissue so as to reduce or eliminate bubbles from vaporizing tissue or blood.

In some examples, a surgical tool 100 includes a balloon 104 including a cavity configured to contain a cooling fluid, in which removing the heat from the electrical conductors includes transmitting the heat from the electrical conductors to the cooling fluid. FIG. 10B is a flow chart of an example method for cooling electrical conductors using a cooling fluid within a balloon 104, in accordance with some examples of the present disclosure. The method of FIG. 10B will be described with respect to FIGS. 1-6, but can be used with other devices and systems described herein. The method of FIG. 10B includes positioning a surgical tool 100, e.g., including balloon 104, in a heart of a patient, such as described in step 1000 of FIG. 10A above (1010). The method of FIG. 10B optionally includes energizing electrical conductors, e.g., for cutting cardiac tissue (1012).

The method of FIG. 10B includes discharging a cooling fluid into a cavity of balloon 104 to remove heat from the electrical conductors into the cooling fluid (1014). For example, a clinician may interact with user interface 35 of control unit 14 or another device, and cause the fluid supply reservoir 16 to deliver the cooling fluid to the cavity of the balloon 104.

In further examples, a radio frequency ablation may be performed by the electrical conductors 106 simultaneous to a cryoablation of the tissue being cut. That is, the steps in the surgical procedure of cutting the tissue and ablating the tissue may be performed together by cryoablating the tissue while cutting, or sequentially by cryoablating the tissue before or after cutting. Furthermore, cryoablation is not the only use for the cooling fluid in the balloon 104. For example, applying cryogenic fluid to the balloon 104 may secure the balloon 104 to tissue via cryoadherence. That is, the balloon may adhere to tissue during a surgical procedure via freezing. In another example, applying cooling fluid to the balloon 104 may provide mechanical stability of the tissue during cutting.

While the illustrated example of FIG. 1 has been described such that the cutting surface of the electrical conductors 106 is at a distal end 112 of the balloon 104, the present disclosure is not limited thereto. That is, in other examples, the cutting surface of the electrical conductors 106 may be at a proximal end 108 of the balloon 104. In still other examples, the cutting surface of the electrical conductors 106 may be both at a distal end 112 and a proximal end 108 of the balloon 104. Accordingly, in an example where the cutting surface is at a distal end 112 of the balloon 104, the cutting operation may be performed by advancing or pushing the elongated support member 102 in a distal direction, e.g., in the direction from the right atrium to the left atrium (towards the right in FIG. 1). In an example where the cutting surface is at a proximal end 108 of the balloon 104, the cutting operation may be performed by retracting or pulling the elongated support member 102 in a proximal direction, e.g., in the direction from the left atrium to the right atrium (towards the left in FIG. 1). In this example, prior to cutting, the balloon 104 may be in a deflated state as it is advanced through the puncture in the interatrial septum. Here, the balloon 104 may be inflated after the elongated support member 102 is advanced from the right atrium into the left atrium of the patient's heart. As in other examples, the cutting operation may be performed after the balloon is fully inflated.

In a further aspect of this disclosure, in some examples, when the balloon 104 is inflated, the electrical conductors 106 may be configured to lie apart from one or both of the distal surface of the balloon 104, and/or a proximal surface of the balloon 104. That is, at least a portion of the electrical conductors 106 (e.g., an exposed portion) may be positioned such that it does not contact the surface of the balloon, or such that there is a space between the exposed portion of the electrical conductors 106 and the surface of the balloon 104. The space between the exposed portion of the electrical conductors 106 and the surface of the balloon 104 may be small, e.g., ranging from about 0.1 mm to 5 mm. For example, FIG. 3 illustrates a surgical tool where a plurality of electrical conductors 106 are configured to lie apart from the proximal surface 108 of the balloon 104. In this example, the portion of the electrical conductors 106 that lies apart from the proximal surface 108 of the balloon 104 is the cutting surface of the electrical conductors. That is, in the illustrated example, the electrical conductors 106 are insulated at a distal surface 112 of the balloon 104, and the surgical tool is configured for cutting while being retracted or pulled (e.g., toward the left in FIG. 3). Accordingly, in some examples, heat from the electrical conductors 106 caused by RF/PFA ablation may be separated from the surface of the balloon 104, protecting the balloon 104 from potential damage. Further, the gap between the balloon and the electrical conductors 106 may further ensure that the force applied from the wire to the tissue and its contact area is increased or maximized.

FIG. 4 shows an illustration of a surgical tool where a plurality of electrical conductors 106 are configured to lie apart from the proximal surface 108 of the balloon 104, and to lie apart from a distal surface 112 of the balloon 104. In this example, the cutting surface of the electrical conductors 106 may be located at both a distal portion 112 of the surgical tool, for cutting as the surgical tool is advanced from the right atrium toward the left atrium, and a proximal portion 108 of the surgical tool, for cutting as the surgical tool is retracted from the left atrium toward the right atrium. In another example (not illustrated), the electrical conductors 106 may lie apart from the distal surface 112 of the balloon, but may lie along a surface of the proximal end 108 of the balloon 104. That is, the electrical conductors 106 may be configured to lie apart from one or both of the distal surface 112 of the balloon, and/or the proximal surface 108 of the balloon.

In the illustrations of FIGS. 3-4, electrical conductors 106 are shown as flexible wires that may flex or bend as the balloon 104 inflates. However, this is not necessarily the case. In other examples (not illustrated), electrical conductors 106 may be rigid electrodes that are actuated outward when the balloon 104 is inflated. Here, when the balloon 104 is inflated, the cutting electrode portion of the electrical conductors 106 can bend or shift outward, such that the rigid electrical conductors lie apart from the surface of the balloon 104. In this example, with rigid electrical conductors deployed with an inflated balloon 104, the surgical tool may be pushed or pulled against the atrial septum for cutting. In this example, the balloon 104 may be larger than the cut size. That is, the balloon 104 may function as a stop, controlling how far the cutter extends through the atrial septum.

FIGS. 5A and 5B illustrate a surgical tool according to a further aspect of this disclosure. In some examples, a surgical tool may include a dual-lobe balloon 502, including a proximal lobe 504 and a distal lobe 506. Between the lobes of the balloon is a relatively narrow waist region 508. Electrical conductors 510 extend longitudinally along the dual-lobe balloon 502 in much the same way as described above and illustrated in FIGS. 1-4 with respect to a single-lobe balloon. However, the electrical conductors 510 lie apart from the surface of the balloon 502 in the region of the waist 508. That is, the electrical conductors 510 may bridge the waist 508 between the balloon lobes, from one lobe to the other.

According to some examples, the electrical conductors 510 may be configured to have a variable, controlled tension as they bridge the waist region of the balloon. For example, the handle 24 (see FIG. 9) may include an actuator for pulling on the wires (e.g., a manual actuator such as a lever or a sliding button; or an automatic actuator such as a spring or tensioning mechanism that keeps a known tension on the electrical conductors 510).

For example, FIG. 5A illustrates a dual-lobe balloon during an atrial shunting procedure, with one of the lobes on each side of the atrial septum 512. That is, to reach the position in the illustration of FIG. 5A, a surgical tool with a deflated balloon may be advanced about halfway through a puncture in the interatrial septum 512, and the balloon 502 may be inflated in place, such that one lobe lies on each side of the atrial septum 512 (i.e., a proximal lobe 504 is in the right atrium while a distal lobe 506 is in the left atrium). In some examples, the proximal lobe 504 and the distal lobe 506 may be configured to separately inflate. That is, there may be separate lumen for introducing an inflating agent into the respective lobes, one at a time in some examples.

In still further examples, the surgical tool may be employed to dilate the puncture opening to create a multi-leaflet, multicuspid valve-like opening in the interatrial septum. For example, in FIG. 5A, the electrical conductors are loose. The dual-lobe balloon 502 is inflated, with a waist region 508 of the balloon lying in a puncture hole in the interatrial septum 512. Here, the electrical conductors 510 may generally conform to the shape of the waist 508 of the dual-lobe balloon 502. Although it is not necessary, in some examples, the waist region 508 of the dual-lobe balloon 502 may have a diameter larger than that of the puncturing tool that created the puncture opening, causing a partial dilation of the puncture opening.

According to an aspect of the present disclosure, the electrical conductors 510 may be activated or energized as a tension is applied across the electrical conductors 510. causing the electrical conductors 510 to radially spread apart from the waist region 508 of the balloon 502 and cut into the atrial septum 512 via RF/PFA ablation. As illustrated in FIG. 5B, a clinician may apply tension to the previously loose electrical conductors 510. For example, the portion 516 of an electrical conductor 510 that is proximal to the waist region 508 may be pulled in a proximal direction; the portion 518 of an electrical conductor 510 that is distal to the waist region 508 may be pulled in a distal direction; or both. As tension is applied to the electrical conductors 510, the portion 514 of the electrical conductors that bridges the waist region of the dual-lobe balloon may tend to straighten out, cutting through septal tissue 512 in proximity to the puncture opening.

In the illustrations of FIGS. 5A-5B, the electrical conductors 510 may be electrically insulated outside the cutting region 514 of the electrical conductors, which spans the waist 508 of the dual-lobe balloon 502. For example, the electrical conductors 510 may have an insulating sheath which is removed or absent in the cutting region 514. In another example, the electrical conductors 510 may be generally flat in shape, and electrically insulated on one side of the flat. That is, the portion of flat electrical conductors that is in contact with the balloon 502 may be insulated while an outer portion of the flat conductors may be exposed or non-insulated, for cutting septal tissue 512.

FIG. 6 is an illustration of another example of a surgical tool for creating an interatrial shunt. The surgical tool of FIG. 6 is similar to that described above in relation to FIG. 5. Here, rather than using a dual-lobe balloon 502, the surgical tool includes two balloons 604 and 606. The two balloons may have an ellipsoid shape, or any suitable shape such that electrical conductors 610 bridge a waist region 608 between the two balloons. Similar to the previous example, the surgical tool of FIG. 6 may be placed such that the distal balloon 606 lies in a left atrium of a patient's heart while the proximal balloon 604 lies in a right atrium, the balloons being separated by the interatrial septum (not illustrated in FIG. 6). Once the balloons 604 and 606 are in place, e.g., sandwiching the interatrial septum, a tension may be applied to the electrical conductors 610. In the illustration of FIG. 6, the electrical conductors 610 are in tension, such that the cutting region 614 of the electrodes 610 lies apart from (e.g., is radially separated away from) the waist region 608.

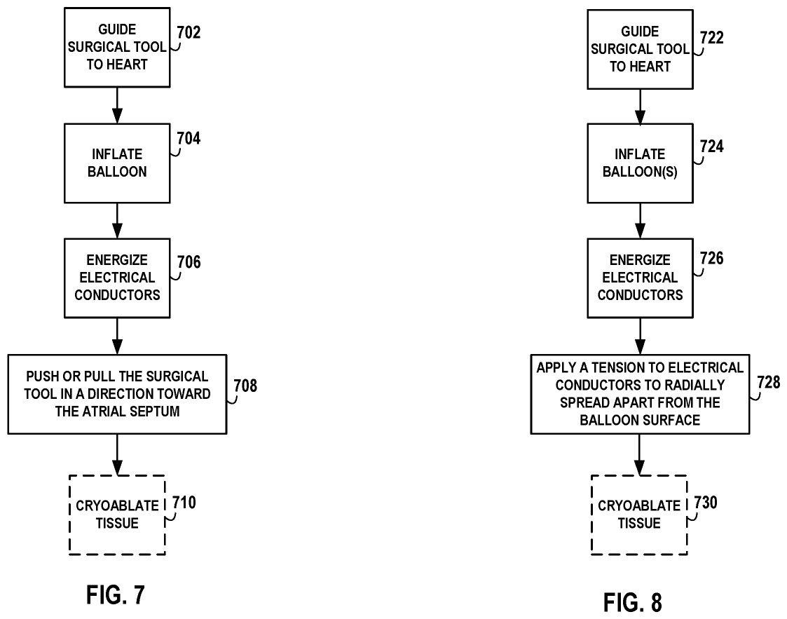

FIG. 7 is a flow chart illustrating an exemplary process for a surgical tool according to one or more aspects of this disclosure. The process of FIG. 7 may be performed to create a multicuspid interatrial shunt. In some examples, the surgical tool referred to in FIG. 7 may be the elongated support member 102 with balloon 104 and a plurality of electrical conductors 106, and may be configured according to any of FIGS. 1, 2A, 2B, 3, and/or 4.

The surgical tool may be guided to a heart of a patient (702). For example, the surgical tool may be configured to fit in a lumen of a steerable catheter, which a clinician may steer or guide through a patient's circulatory system into the patient's heart. In another example, the surgical tool may include a guidewire lumen, such that a guidewire may be used to guide the surgical tool through the patient's circulatory system into the patient's heart.

The surgical tool may inflate a balloon 104 at its distal end (e.g., within the patient's heart) (704). Accordingly, the electrical conductors 106, which extend longitudinally along the balloon 104 and are radially spaced apart from one another on a surface of the balloon 104, may flex or bend such that the electrical conductors 106 expand in a radial direction when the balloon 104 is inflated.

The surgical tool may energize the electrical conductors 106 (706). That is, electrical energy may be applied to the electrical conductors 106 such that the electrical conductors 106 can cut the tissue of the atrial septum.

The surgical tool may then be pushed or pulled (708) in a direction toward the atrial septum to bring the exposed portion of the electrical conductors 106 into contact with the atrial septum. For example, in an example where a distal surface 112 of the balloon 104 is a cutting surface with exposed portions of the electrical conductors 106, the surgical tool may be located in the right atrium and pushed or advanced to position the surgical tool forward toward the atrial septum. On the other hand, in an example where a proximal surface 108 of the balloon 104 is a cutting surface, the surgical tool may be located in the left atrium and pulled or retracted to position the surgical tool back toward the atrial septum. By moving the surgical tool into the atrial septum, the exposed portions of the electrical conductors 106 may cut the tissue of the atrial septum, forming a multicuspid opening in the atrial septum.

As discussed above, in some examples, the surgical tool 100 may cryoablate tissue (710). In the example of FIG. 7, cryoablation of the tissue after cutting can stabilize the tissue and provide for a more robust shunt in the atrial septum. Although the process of FIG. 7 shows cryoablation taking place after the cutting phase at step 708, aspects of this disclosure are not limited thereto. That is, in various examples block 710 may appear before block 708, after block 708, or blocks 708 and 710 may be performed simultaneously.

FIG. 8 is a flow chart illustrating an exemplary process for a surgical tool according to further aspects of this disclosure. Like the process of FIG. 7, the process of FIG. 8 may be performed to create a multicuspid interatrial shunt. In some examples, the surgical tool referred to in FIG. 8 may be an elongated support member with electrical conductors and a dual-lobe balloon 502 as illustrated in FIGS. 5A and 5B. In some examples, the surgical tool referred to in FIG. 8 may be an elongated support member with electrical conductors and two balloons 604 and 606 as illustrated in FIG. 6.

The surgical tool may be guided to a heart of a patient (722). For example, the surgical tool may be configured to fit in a lumen of a steerable catheter, which a clinician may steer or guide through a patient's circulatory system into the patient's heart. In another example, the surgical tool may include a guidewire lumen, such that a guidewire may be used to guide the surgical tool through the patient's circulatory system into the patient's heart. In this example, the surgical tool may be positioned within the heart such that the balloon's separate lobes are on different sides of the atrial septum. That is, a dual-lobe balloon 502 may be advanced such that a distal lobe passes through a puncture in the atrial septum into the left atrium, while a proximal lobe remains in the right atrium. In a two-balloon example, as in FIG. 6, the distal balloon or lobe 606 may be advanced to pass through a puncture in the atrial septum into the left atrium, while a proximal balloon or lobe 604 remains in the right atrium.

The surgical tool may inflate the one or more balloons 502 or 604 and 606 (724). Accordingly, the electrical conductors 106, which extend longitudinally along the balloon 502 or the balloons 604 and 606 and are radially spaced apart from one another on a surface of the balloon(s) may flex, such that the electrical conductors 106 expand in a radial direction when the balloon 502 or the balloons 604 and 606 are inflated. In this example, when the balloon or balloons are inflated, an exposed portion of the electrical conductors 106 at least partially bridges a waist region between the balloon's lobes. According to an aspect of this disclosure, as the balloon(s) are inflated, the wires or electrical conductors 106 may be loose, such that they generally conform to the surface of the waist region between the balloon's lobes.

The surgical tool may energize the electrical conductors 106 (726). That is, electrical energy may be applied to the electrical conductors 106 such that the electrical conductors 106 can cut the tissue of the atrial septum.

The surgical tool may apply a tension (728) to the electrical conductors 106 to radially spread them apart from the surface of the balloon 104 (e.g., from the waist region between the balloon's lobes). As the electrical conductors 106 radially spread apart from the waist region, because the waist region lies in a puncture opening in the atrial septum, the electrical conductors 106 may cut tissue of the atrial septum, forming a multicuspid opening in the atrial septum.

As discussed above, in some examples, the surgical tool 100 may cryoablate tissue (730). In the example of FIG. 8, cryoablation of the tissue after cutting can stabilize the tissue and provide for a more robust shunt in the atrial septum. Although the process of FIG. 8 shows cryoablation taking place after the cutting phase at step 728, aspects of this disclosure are not limited thereto. That is, in various examples block 730 may appear before block 728, after block 728, or blocks 728 and 730 may be performed simultaneously.

Although example systems and techniques have been shown and described, it is to be understood that all the terms used herein are descriptive rather than limiting, and that many changes, modifications, and substitutions may be made by one having ordinary skill in the art without departing from the spirit and scope of the invention. The following examples are examples of systems, devices, and methods described herein.

Example 1: in some examples, a medical device includes an elongated support member, an inflatable balloon at a distal portion of the elongated support member, and a plurality of electrical conductors extending longitudinally along the balloon. The plurality of conductors are configured to deliver electrical energy to an atrial septum of a patient, the electrical conductors being radially spaced apart from one another to form a multicuspid opening in the atrial septum by delivery of the electrical energy.

Example 2: in some examples of the medical device of Example 1, the electrical conductors lie along an outer surface of the inflatable balloon, extending from a distal end of the inflatable balloon to a proximal end of the inflatable balloon.

Example 3: in some examples of the medical device of Examples 1 or 2, when the balloon is inflated the electrical conductors are configured to lie apart from a distal surface of the balloon such that the electrical conductors do not contact a portion of the distal surface of the balloon.

Example 4: in some examples of the medical device of any of Examples 1 to 3, when the balloon is inflated the electrical conductors are configured to lie apart from a proximal surface of the balloon such that the electrical conductors do not contact a portion of the proximal surface of the balloon.

Example 5: in some examples of the medical device of any of Examples 1 to 4, the medical device further includes a puncturing element at a distal tip of the elongated support member, the puncturing clement configured to puncture the atrial septum.

Example 6: in some examples of the medical device of any of Examples 1 to 5, at least one of the electrical conductors extends to a distal tip of the elongated support member, wherein the at least one of the electrical conductors is configured to provide electrical energy to the distal tip for puncturing the atrial septum.

Example 7: in some examples of the medical device of any of Examples 1 to 6, the electrical conductors include a flexible material such that the electrical conductors expand in a radial direction when the inflatable balloon is inflated.

Example 8: in some examples of the medical device of any of Examples 1 to 7, the inflatable balloon includes a distal lobe and a proximal lobe with a waist between the distal lobe and the proximal lobe, wherein the electrical conductors bridge the waist between the distal lobe and the proximal lobe, and wherein the electrical conductors are configured to expand in a radial direction away from the waist when tension is applied to the electrical conductors.

Example 9: in some examples of the medical device of any of Examples 1 to 8, the inflatable balloon is a cryoablation balloon configured for cryoablating tissue with a cryogenic agent.

Example 10: in some examples of the medical device of any of Examples 1 to 9, the inflatable balloon is configured to be at least partially filled with a cooling agent for cooling surrounding blood or tissue when the electrical conductors are energized.

Example 11: in some examples of the medical device of any of Examples 1 to 10, the plurality of electrical conductors consists of three electrical conductors radially spaced apart by about 120° such that the multicuspid opening is a tri-leaflet opening.

Example 12: in some examples of the medical device of any of Examples 1 to 11, the plurality of electrical conductors consists of four electrical conductors radially spaced apart by about 90° such that the multicuspid opening is a four-leaflet opening.

Example 13: in some examples of the medical device of any of Examples 1 to 12, the elongated support member includes a guidewire lumen extending through its length such that the elongated support member can be guided via a guidewire.

Example 14: in some examples of the medical device of any of Examples 1 to 13, wherein the plurality of electrical conductors are each insulated along their length, being at least partially exposed at one or both of a distal surface of the inflatable balloon or a proximal surface of the inflatable balloon.

Example 15: in some examples, a method includes guiding a surgical tool to a heart of a patient, inflating at least one balloon at a distal end of the surgical tool, energizing a plurality of electrical conductors that extend longitudinally along the balloon, the electrical conductors being radially spaced apart from one another on a surface of the at least one balloon, and forming a multicuspid opening in an atrial septum of the patient by delivering electrical energy to the atrial septum via the electrical conductors.

Example 16: in some examples of the method of Example 15, the plurality of electrical conductors include an insulated portion and an exposed portion. The method further includes pushing or pulling the surgical tool in a direction toward the atrial septum to bring the exposed portion of the electrical conductors into contact with the atrial septum.

Example 17: in some examples of the method of Example 15, wherein the at least one balloon comprises a distal lobe and a proximal lobe with a waist region between the distal lobe and the proximal lobe, wherein the plurality of electrical conductors comprise an insulated portion, and an exposed portion at least partially bridging the waist region between the distal lobe and the proximal lobe. The method further includes applying tension to the plurality of electrical conductors to cause the electrical conductors to radially spread apart from the waist region to cut the multicuspid opening in the atrial septum.

Example 18: in some examples of the method of any of Examples 15 to 17, the method further includes applying a cryogenic fluid to the at least one balloon.

Example 19: in some examples of the method of any of Examples 15 to 18, applying the cryogenic fluid to the at least one balloon includes at least one of: cooling the balloon to reduce heat on the balloon from the electrical conductors; cooling tissue or surrounding blood to reduce bubbles from vaporizing the tissue or the surrounding blood; securing balloon contact to tissue via cryoadherence; or providing mechanical stability of the tissue during cutting.

Example 20: in some examples of the method of any of Examples 15 to 18, applying the cryogenic fluid to the at least one balloon includes cryoablating tissue of the atrial septum.

The techniques described in this disclosure may be implemented, at least in part, in hardware, software, firmware or any combination thereof. For example, various aspects of the described techniques may be implemented within one or more processors or processing circuitry, including one or more microprocessors, digital signal processors (DSPs), application specific integrated circuits (ASICs), field programmable gate arrays (FPGAs), or any other equivalent integrated or discrete logic circuitry, as well as any combinations of such components. The term “processor” or “processing circuitry” may generally refer to any of the foregoing logic circuitry, alone or in combination with other logic circuitry, or any other equivalent circuitry. A control unit comprising hardware may also perform one or more of the techniques of this disclosure.

Such hardware, software, and firmware may be implemented within the same device or within separate devices to support the various operations and functions described in this disclosure. In addition, any of the described units, circuits or components may be implemented together or separately as discrete but interoperable logic devices. Depiction of different features as circuits or units is intended to highlight different functional aspects and does not necessarily imply that such circuits or units must be realized by separate hardware or software components. Rather, functionality associated with one or more circuits or units may be performed by separate hardware or software components or integrated within common or separate hardware or software components.

The techniques described in this disclosure may also be embodied or encoded in a computer-readable medium, such as a computer-readable storage medium, containing instructions that may be described as non-transitory media. Instructions embedded or encoded in a computer-readable storage medium may cause a programmable processor, or other processor, to perform the method, e.g., when the instructions are executed. Computer readable storage media may include random access memory (RAM), read only memory (ROM), programmable read only memory (PROM), erasable programmable read only memory (EPROM), electronically erasable programmable read only memory (EEPROM), flash memory, a hard disk, a CD-ROM, a floppy disk, a cassette, magnetic media, optical media, or other computer readable media.

Various examples have been described. These and other examples are within the scope of the following claims.

1. A medical device comprising:
an elongated support member;
an inflatable balloon at a distal portion of the elongated support member; and
a plurality of electrical conductors extending longitudinally along the balloon, the plurality of conductors configured to deliver electrical energy to an atrial septum of a patient, the electrical conductors being radially spaced apart from one another to form a multicuspid opening in the atrial septum by delivery of the electrical energy.

2. The medical device of claim 1, wherein the electrical conductors lie along an outer surface of the inflatable balloon, extending from a distal end of the inflatable balloon to a proximal end of the inflatable balloon.

3. The medical device of claim 1, wherein when the balloon is inflated the electrical conductors are configured to lie apart from a distal surface of the balloon such that the electrical conductors do not contact a portion of the distal surface of the balloon.

4. The medical device of claim 1, wherein when the balloon is inflated the electrical conductors are configured to lie apart from a proximal surface of the balloon such that the electrical conductors do not contact a portion of the proximal surface of the balloon.

5. The medical device of claim 1, further comprising a puncturing element at a distal tip of the elongated support member, the puncturing element configured to puncture the atrial septum.

6. The medical device of claim 1, wherein at least one of the electrical conductors extends to a distal tip of the elongated support member, wherein the at least one of the electrical conductors is configured to provide electrical energy to the distal tip for puncturing the atrial septum.

7. The medical device of claim 1, wherein the electrical conductors comprise a flexible material such that the electrical conductors expand in a radial direction when the inflatable balloon is inflated.

8. The medical device of claim 1, wherein the inflatable balloon comprises a distal lobe and a proximal lobe with a waist between the distal lobe and the proximal lobe,
wherein the electrical conductors bridge the waist between the distal lobe and the proximal lobe, and
wherein the electrical conductors are configured to expand in a radial direction away from the waist when tension is applied to the electrical conductors.

9. The medical device of claim 1, wherein the inflatable balloon is a cryoablation balloon configured for cryoablating tissue with a cryogenic agent.

10. The medical device of claim 1, wherein the inflatable balloon is configured to be at least partially filled with a cooling agent for cooling surrounding blood or tissue when the electrical conductors are energized.

11. The medical device of claim 1, wherein the plurality of electrical conductors consists of three electrical conductors radially spaced apart by about 120° such that the multicuspid opening is a tri-leaflet opening.

12. The medical device of claim 1, wherein the plurality of electrical conductors consists of four electrical conductors radially spaced apart by about 90° such that the multicuspid opening is a four-leaflet opening.

13. The medical device of claim 1, wherein the elongated support member comprises a guidewire lumen extending through its length such that the elongated support member can be guided via a guidewire.

14. The medical device of claim 1, wherein the plurality of electrical conductors are each insulated along their length, being at least partially exposed at one or both of a distal surface of the inflatable balloon or a proximal surface of the inflatable balloon.

15. A method comprising:
guiding a surgical tool to a heart of a patient;
inflating at least one balloon at a distal end of the surgical tool;
energizing a plurality of electrical conductors that extend longitudinally along the balloon, the electrical conductors being radially spaced apart from one another on a surface of the at least one balloon; and
forming a multicuspid opening in an atrial septum of the patient by delivering electrical energy to the atrial septum via the electrical conductors.

16. The method of claim 15, wherein the plurality of electrical conductors comprise an insulated portion and an exposed portion,
the method further comprising: pushing or pulling the surgical tool in a direction toward the atrial septum to bring the exposed portion of the electrical conductors into contact with the atrial septum.

17. The method of claim 15,
wherein the at least one balloon comprises a distal lobe and a proximal lobe with a waist region between the distal lobe and the proximal lobe,
wherein the plurality of electrical conductors comprise an insulated portion, and an exposed portion at least partially bridging the waist region between the distal lobe and the proximal lobe,
the method further comprising: applying tension to the plurality of electrical conductors to cause the electrical conductors to radially spread apart from the waist region to cut the multicuspid opening in the atrial septum.

18. The method of claim 15, further comprising applying a cryogenic fluid to the at least one balloon.

19. The method of claim 18, wherein applying the cryogenic fluid to the at least one balloon comprises at least one of:
cooling the balloon to reduce heat on the balloon from the electrical conductors;
cooling tissue or surrounding blood to reduce bubbles from vaporizing the tissue or the surrounding blood;
securing balloon contact to tissue via cryoadherence; or
providing mechanical stability of the tissue during cutting.

20. The method of claim 18, wherein applying the cryogenic fluid to the at least one balloon comprises cryoablating tissue of the atrial septum.
